(19) United States
(12) Patent Application Publication
(10) Pub. No.: US 2019/0134274 A1
(43) Pub. Date: May 9, 2019

(54) ENGINEERING MULTILEVEL CELL SHEET-DERIVED BLOOD VESSELS

(71) Applicant: The Board of Trustees of the Leland Stanford Junior University, Stanford, CA (US)

(72) Inventors: Daniel von Bornstaedt, Wernigerode (DE); Y. Joseph Woo, Menlo Park, CA (US); Hanjay Wang, Palo Alto, CA (US)

(21) Appl. No.: 16/180,359

(22) Filed: Nov. 5, 2018

Related U.S. Application Data
(60) Provisional application No. 62/584,088, filed on Nov. 9, 2017.

(51) Int. Cl. A61L 27/50; A61L 27/48; A61L 27/36; A01N 1/02; A61L 27/38; C12N 5/077

(52) U.S. Cl. CPC A61L 27/507; C12N 2502/1329; A61L 27/3691; A61L 27/3625; A01N 1/021; A61L 27/3895; A61L 27/3886; A61L 27/3826; A61L 27/3808; A61L 27/3604; C12N 5/0656; C12N 5/0661; C12N 5/0657; A61L 2430/40; A61L 2430/20; C12N 2502/1323; C12N 2502/1347; A61L 27/48

(57) ABSTRACT

Engineered multilevel cell sheet-derived blood vessels and methods of preparing and using them are disclosed. Blood vessels are generated by wrapping cell sheets around a rod-like device, such as an angiocath needle, to form a tube, which is stabilized with a cyanoacrylate membrane or fibrin glue followed by endothelialization. Such engineered blood vessels can be implanted in tissue and used in vascular surgery as vascular bypass or interposition grafts as well as for vascularization and perfusion of tissue or organs prior to transplant.

### CROSS REFERENCE

This application claims benefit of U.S. Provisional Patent Application No. 62/584,088, filed Nov. 9, 2017, which application is incorporated herein by reference in its entirety.

### FEDERALLY SPONSORED RESEARCH AND DEVELOPMENT

This invention was made with Government support under contract HL089315 awarded by the National Institutes of Health. The Government has certain rights in the invention.

### TECHNICAL FIELD

The present invention pertains generally to tissue engineering of blood vessels. In particular, the invention relates to engineered multilevel cell sheet-derived blood vessels and methods of generating and using such engineered blood vessels in vascular surgery.

### BACKGROUND

Cardiovascular diseases are the main cause of death in the United States. Bypass grafting is one of the most frequently indicated treatment options particularly for complex cases of coronary and peripheral artery disease. Preferably, a patient's own vein or artery is used as a bypass vessel. If this option is not available (due to multiple bypass procedures or vascular degeneration), an artificial bypass graft is needed. However, there is a significant difference in clinical outcome between an autologous and an artificial vessel graft, the patency rates after two years being above 90% for a saphenous vein graft as opposed to 32% for a polytetrafluoroethylene derived grafts. Therefore, current research focuses on biological alternatives to artificial bypass grafts.

So far two approaches have been used to engineer biological vessel grafts: i) based on a scaffold or ii) cell-only approaches. While scaffold based vessel grafts tend to have significantly reduced patency rates, the production process for cell-only grafts is time consuming (6 weeks to several months) and work intense.

There remains a need for improved methods for engineering biological vessel grafts to treat cardiovascular diseases.

### SUMMARY

Provided are compositions and methods for engineered multilevel cell sheet-derived blood vessels; and methods of generating and using such engineered blood vessels in vascular surgery.

In one aspect, a method of making a tissue-engineered blood vessel is provided, comprising: a) culturing fibroblasts and smooth muscle cells in vitro to form one or more confluent cell sheets; b) wrapping said one or more cell sheets around a rod-like device to form a tube; c) stabilizing the tube formed from the cell sheets with a cyanoacrylate membrane or fibrin glue; d) endothelialization of the tube formed from the cell sheets by culturing with endothelial cells; and e) removing the rod-like device to form the tissue-engineered blood vessel.

In one embodiment, the fibroblasts and smooth muscle cells are from a human subject. In certain embodiments, the endothelial cells are vascular endothelial cells. In another embodiment, the endothelial cells are human umbilical vein endothelial cells.

In certain embodiments, the rod-like device is a needle or mandrel. For example, an angiocath needle may be used having a gauge of at least 11, at least 16, at least 18, at least 20, at least 22, or at least 22.5; and up to about a gauge of 24. In certain embodiments, the needle has a gauge ranging from 11 to 24, e.g. such as a 11, 16, 18, 20, 22, 22.5, or 24 gauge needle.

In certain embodiments, a plurality of cell sheets, which may be of from about 1 cm in diameter, up to about 10 cm in diameter, e.g. about 2, 3, 4, 5, 6, 7, 8, 9, cm in diameter, or layers of cells from a single sheet are wrapped around the rod-like device, for example at least about 4, at least about 5, at least about 6, at least about 7, at least about 8, at least about 9, at least about 10, at least about 11, and up to about 12 cell sheets are wrapped around the rod-like device to form the tube. In certain embodiments, 5 to 10 cell sheets are wrapped around the rod-like device to form the tube, including any number of cell sheets within this range, such as 5, 6, 7, 8, 9, or 10 cell sheets. In another embodiment, the diameter of the rod-like device is less than or equal to 1 mm.

In another aspect, a tissue-engineered blood vessel produced as described herein is provided. In certain embodiments, the inner diameter of the tissue-engineered blood vessel is less than or equal to 1 mm.

In another aspect, a method of treating a subject for a cardiovascular disease or disorder is provided, the method comprising implanting a tissue-engineered blood vessel, as described herein, in the subject. In another embodiment, the method further comprises linking the tissue-engineered blood vessel to a vein or artery by surgical anastomosis. In certain embodiments, a tissue-engineered blood vessel, as described herein, is used as a vascular bypass or interposition graft.

In another embodiment, a tissue-engineered blood vessel, as described herein, is used in treatment of a cardiovascular disease or disorder, such as, but not limited to coronary artery disease, ischemic heart disease, ischemic stroke, peripheral artery disease, cerebrovascular disease, atherosclerosis, arteriosclerosis, angina, myocardial infarction, and embolism. In certain embodiments, the fibroblasts, smooth muscle cells, or endothelial cells are autologous, xenogeneic, or allogeneic.

In another aspect, a method of vascularizing a tissue or organ for transplant is provided, the method comprising implanting a tissue-engineered blood vessel, as described herein, in the tissue or organ. In one embodiment, the tissue is heart muscle. Implantation of the tissue-engineered blood vessel may be performed prior to or after transplant of the tissue or organ into a subject. In another embodiment, the method further comprises cultivation of the tissue around the tissue-engineered blood vessel.

In another embodiment, the method further comprises perfusing the vascularized tissue wherein blood flows through the tissue-engineered blood vessel. In another embodiment, the method further comprises transplanting the vascularized tissue or organ into a subject. In another embodiment, the method further comprises linking the tissue-engineered blood vessel to a vein or artery by surgical anastomosis.

In another aspect, a method of engineering a perfused heart muscle tissue graft is provided, the method comprising: a) producing a tissue-engineered blood vessel, as described herein; b) co-culturing the tissue-engineered blood vessel with cardiomyocytes to produce a vascularized heart muscle tissue graft, wherein vessel sprouting from the tissue-engineered blood vessel produces a vascular network within the heart muscle tissue graft; and c) perfusing the cardiomyocyte tissue graft, wherein blood flows through the tissue-engineered blood vessel and the vascular network within the heart muscle tissue graft.

In certain embodiments, the cardiomyocytes are autologous, xenogeneic, or allogeneic. In another embodiment, the cardiomyocytes are human induced cardiomyocytes.

These and other embodiments of the subject invention will readily occur to those of skill in the art in view of the disclosure herein.

### BRIEF DESCRIPTION OF THE FIGURES

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1D show construction and evaluation of cell-sheet derived engineered vessels. FIG. 1A shows an engineered vessel. FIG. 1B shows a cell-sheet engineered vessel anastomosed to a host femoral artery. FIG. 1C shows a hematoxylin and eosin (H&E) stained vessel after 8 weeks in vivo. FIG. 1D shows immunohistology of a vessel after 8 weeks in vivo, smooth muscle actin (green), von Willebrand factor (red), nuclei (blue. DAPI). EVC: Engineered Vascular Conduit.

FIG. 2A-2C show an evaluation of engineered vessels in a nude rat model of hindlimb ischemia. FIG. 2A shows laser doppler images 8 weeks after bilateral femoral artery ligation demonstrating improved perfusion with engineered vessel interposition graft versus control. FIG. 2B and FIG. 2C show blood flow ratios between legs after femoral graft implantation and contralateral ligation or sham surgery (FIG. 2B: Laser Doppler. FIG. 2C: invasive flow probe).

FIG. 3A-3C show engineering and transplantation of vascularized heart tissue. FIG. 3A shows formation of a capillary network in hydrogel containing HUVEC. FIG. 3B shows a light microscopy image demonstrating capillary sprouting into cardiomyocyte culture. FIG. 3C shows surgical anastomosis of an engineered vessel to the left common carotid artery and right ventricle.

FIG. 4A-4F illustrates the process of generating engineered vessels.

FIG. 5 illustrates a perfusion apparatus for the engineered vessels.

### DETAILED DESCRIPTION

The practice of the present invention will employ, unless otherwise indicated, conventional methods of medicine, cell biology, chemistry, biochemistry, molecular biology and recombinant DNA techniques, and immunology, within the skill of the art. Such techniques are explained fully in the literature. See, e.g., R. O. Bonow, D. L. Mann, D. P. Zipes, P. Libby Braunwald's Heart Disease: A Textbook of Cardiovascular Medicine (Saunders; 9^{th} edition, 2011); J. Watchie Cardiovascular and Pulmonary Physical Therapy: A Clinical Manual (Saunders, 2^{nd} edition, 2009); G. Vunjak-Novakovic and R. I. Freshney Culture of Cells for Tissue Engineering (Wiley-Liss, 1^{st} edition, 2006); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); and Sambrook et al., Molecular Cloning: A Laboratory Manual (3^{rd} Edition, 2001).

All publications, patents and patent applications cited herein, whether supra or infra, are hereby incorporated by reference in their entireties.

### DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a cell” includes a mixture of two or more cells, and the like.

The term “about,” particularly in reference to a given quantity, is meant to encompass deviations of plus or minus five percent.

As used herein, the term “cell viability” refers to a measure of the amount of cells that are living or dead, based on a total cell sample. High cell viability, as defined herein, refers to a cell population in which greater than 85% of all cells are viable, preferably greater than 90-95%, and more preferably a population characterized by high cell viability containing more than 99% viable cells.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Transplant” refers to the transfer of a cell, tissue, or organ to a subject from another source.

“Cardiovascular diseases and disorders” include, but are not limited to, coronary artery disease, ischemic heart disease, ischemic stroke, peripheral artery disease, cerebrovascular disease, atherosclerosis, arteriosclerosis, angina, myocardial infarction, and embolism.

“Substantially purified” refers to isolation of a substance or cell (e.g., fibroblasts, smooth muscle cells, or endothelial cells) such that the substance or cell comprises the majority percent of the sample in which it resides. Typically in a sample, a substantially purified component comprises 90%-92%, 93-95%, 96%-98%, or 99%-100% of the sample or any percent within these ranges, including at least 90%, 91%, 92%, 93%, 94%, 95%, 96%, 97%, 98%, 99%, or 100% of the sample. Techniques for purifying cells of interest are well-known in the art and include, for example, fluorescence-activated cell sorting (FACS), magnetic-activated cell sorting (MACS), single cell sorting, affinity chromatography, microfluidic cell separation, and sedimentation according to density.

The terms “subject,” “individual” or “patient” are used interchangeably herein and refer to a vertebrate, preferably a mammal. By “vertebrate” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

### Modes

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The development of a method for engineering multilevel cell sheet-derived blood vessels is provided. In particular, t such vessels were produced from cultures of human fibroblasts and smooth muscle cells. Confluent cell sheets were wrapped around an angiocath needle and stabilized using a cyanoacrylate membrane followed by endothelialization with human umbilical vein endothelial cells. Vessels with inner diameters as small as 1 mm in size were made by these methods. It is further shown that such engineered vessels can be successfully anastomosed to a femoral artery as an interpositional vessel graft (see Examples).

In order to further an understanding of the invention, a more detailed discussion is provided below regarding engineered multilevel cell sheet-derived blood vessels and their use in treatment of cardiovascular diseases and disorders.

### Engineering Blood Vessels from Multilevel Cell Sheets

The method for producing engineered blood vessels typically comprises wrapping cell sheets around a rod-like device to form a tube, which is stabilized with a membrane. The cell sheet-formed tube is then endothelialized to generate an engineered blood vessel. Such engineered blood vessels can be used in vascular surgery as vascular bypass or interposition grafts as well as for vascularization and perfusion of transplant tissue or organs.

The fibroblasts, smooth muscle cells, and endothelial cells used to form the engineered blood vessels can be obtained directly from the subject undergoing treatment, or a donor, a culture of cells from a donor, or from established cell culture lines. The cells may be obtained from the same or a different species than the subject to be treated, but preferably are of the same species, and more preferably of the same immunological profile as the subject. Such cells can be obtained, for example, from a tissue sample (e.g. biopsy of skin, smooth muscle, or blood vessels) collected from the subject to be treated, or a relative or matched donor. Cells obtained from a donor may be treated (e.g., chemically or enzymatically) to remove surface antigens to avoid tissue rejection.

The fibroblasts and smooth muscle cells can be cultured to produce a cohesive sheet using cell sheet tissue engineering approaches known in the art. (see, e.g., Heureux et al. (2006) Nat. Med. 12(3):361-365 and U.S. Patent Application No. 2012/0141547; herein incorporated by reference). For example, fibroblasts and smooth muscle cells can be seeded in two layers on cell culture media on the surface of a solid support (e.g., culture dish or plate). The cells are seeded in two layers: one confluent layer of fibroblasts and after 24 hours another confluent layer of smooth muscle cells. The culture dish has a temperature respondent surface that changes from hydrophobic to hydrophilic upon temperature change so that the cell sheets can be lifted after another 24 hours without scraping or other mechanical manipulation. The cells are organized in two layers when lifted. However, during the wrapping process around the mandrel or needle the cell layers overlap so that there is a mix of cells in the vessel wall. The cells typically form a confluent cell sheet that can be removed from the solid support after about 24 hours (see Examples). The cell sheets have sufficient mechanical strength to be detached and rolled.

Cell sheets are wrapped around a rod-like device to form a tube. One cell sheet may wrapped several around the needle several times; and the cell sheets may partwise overlap so that the wall of the tube consists of 7-10 cell layers. The rod-like device is typically a needle or mandrel, but can be any rod-like device suitable for producing a desired vessel size. In certain embodiments, the rod-like device has a diameter between about 0.5 mm and about 6 mm, including any diameter within this range, such as about 0.5 mm, 0.75 mm, 0.9 mm, 1 mm, 1.5 mm, 2 mm, 3 mm, 4 mm, 5 mm or 6 mm. For example, an angiocath needle may be used for wrapping the cell sheets. In certain embodiments, a needle having a gauge of at least 11, at least 12, at least 14, at least 16, at least 18, at least 20, at least 22, or at least 22.5 is used for wrapping cell sheets. In certain embodiments, the rod-like device is a needle having a gauge ranging from about 11 to about 24, such as a gauge of 11, 12, 14, 16,18, 20, 22, 22.5, or 24.

Those of skill in the art will appreciate that the required diameter will be determined based on the intended use of the engineered vessel. The inner diameter of the engineered vessel is equal to the outer diameter of the needles, i.e. from about 11 to about 24 G. The outer diameter will vary depending on the number of cell layers, and may represent a wall thickness of from about 0.2 mm, about 0.5 mm, about 0.75 mm, about 1 mm, about a 25 mm, etc., such that, for example, a vessel with an inner diameter of 1 mm may have an outer diameter of about 2 mm. The length of the vessel may vary with the specific requirements, e.g. at least about 1 cm in length, at least about 2 cm in length, at least about 5 cm in length, or more as required.

The engineered vessels may be made from a plurality of cells sheets by stacking a plurality of cells sheets on top of each other before wrapping around the rod-like device. In certain embodiments, 5 to 10 cell sheets are wrapped around the rod-like device to form the tube, including any number of cell sheets within this range, such as 5, 6, 7, 8, 9, or 10 cell sheets.

The cell sheet-formed tube can be stabilized by wrapping a membrane around it. For example, a cyanoacrylate membrane can be used for this purpose. A drop of from about 25-100 μl of Dermabond (commercially available tissue sealant) can be dropped onto a media surface, which results in a thin glue membrane. This membrane is wrapped several times around the cell sheet construct. Alternatively, the cell sheet-formed tube can be stabilized with a fibrin glue.

Endothelialization is performed by internal perfusion of the cell sheet-formed tube with endothelial cells and endothelial cell growth media (e.g., typically for at least 1 day). Endothelial cells are provided at a concentration of at least about 10⁴ cells/ml, at least about 10⁵ cells/ml, and usually at a concentration of around 5×10⁵/ml to about 5×10⁶/ml, e.g. around 10⁶/ml. For example, vascular endothelial cells such as from a vein or artery may be used for this purpose, such as HUVECs, etc. After endothelialization, the engineered vessels may be further perfused with growth media (e.g., typically another 10-14 days) to stabilize the vessel wall. The engineered vessels can then be removed from the rod-like device and used in various applications, typically immediately after removing from perfusion.

### Applications

Engineered blood vessels may be implanted in tissue and used to replace blood vessels damaged by a disease or traumatic injury. For example, engineered blood vessels can be used for treating a subject for a cardiovascular disease or disorder such as, but not limited to, coronary artery disease, ischemic heart disease, ischemic stroke, peripheral artery disease, cerebrovascular disease, atherosclerosis, arteriosclerosis, angina, myocardial infarction, and embolism.

In particular, engineered blood vessels can be used in vascular surgery to replace natural veins or arteries. Surgical anastomosis can be performed to connect engineered blood vessels to arteries or veins in the vascular circulation of a subject. For example, engineered blood vessels may be used as vascular bypass grafts to route blood flow around an area of blockage, such as caused by coronary or peripheral arterial disease. Alternatively, engineered blood vessels may be used as interposition grafts to replace damaged segments of vessels that are removed from a subject. Such interposition grafts with engineered blood vessels can be used, for example, to repair a blocked artery or vein or an aneurysm.

In addition, engineered blood vessels may be used in vascular anastomoses of organ transplants. For example, engineered blood vessels can be implanted in an organ to vascularize and perfuse tissue prior to or after transplant.

### Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Engineering Multi-Level Cell Sheet Derived Human Vessel Grafts

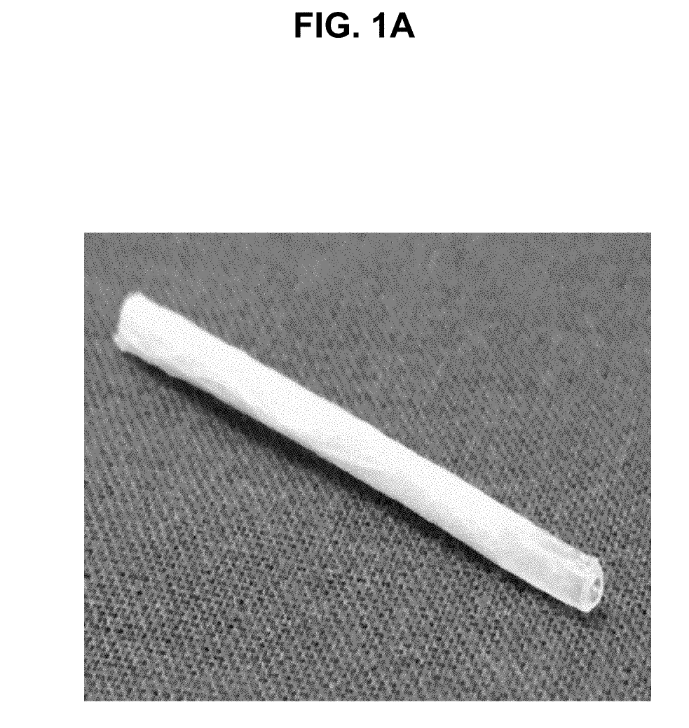

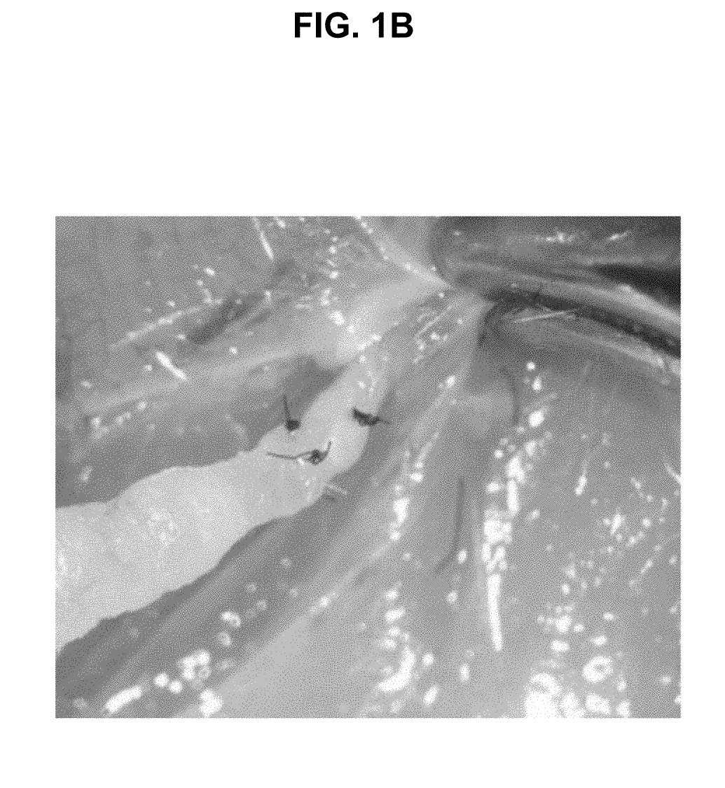

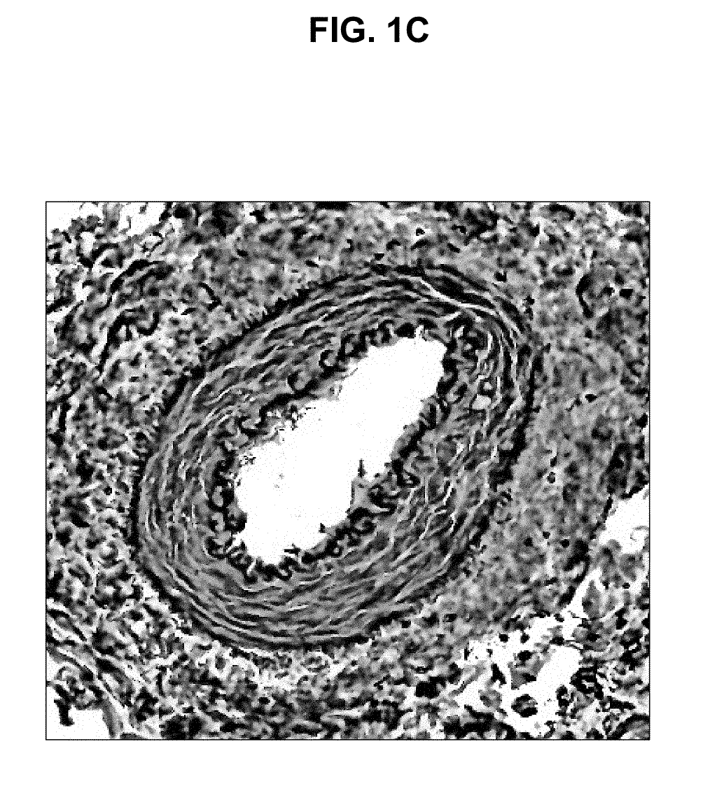

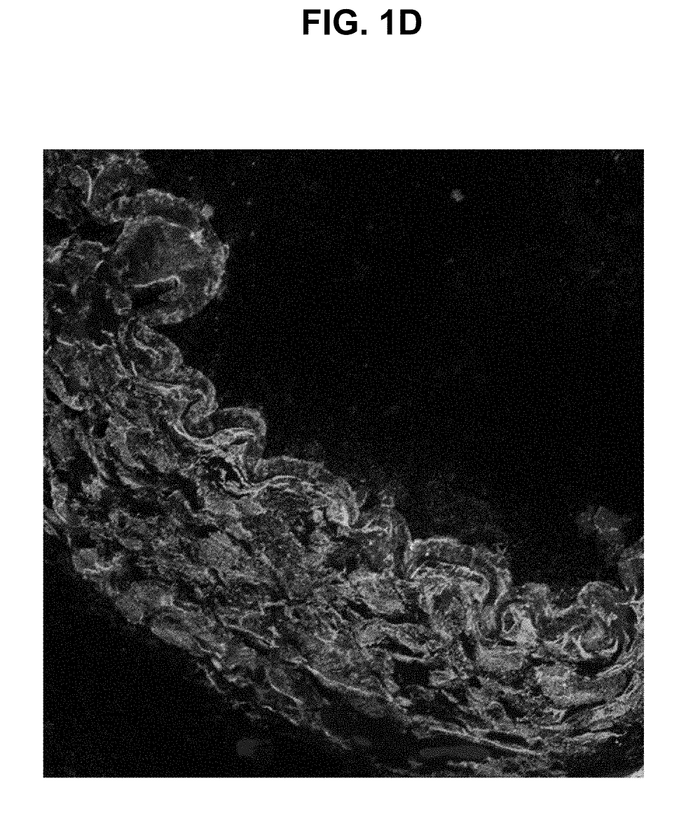

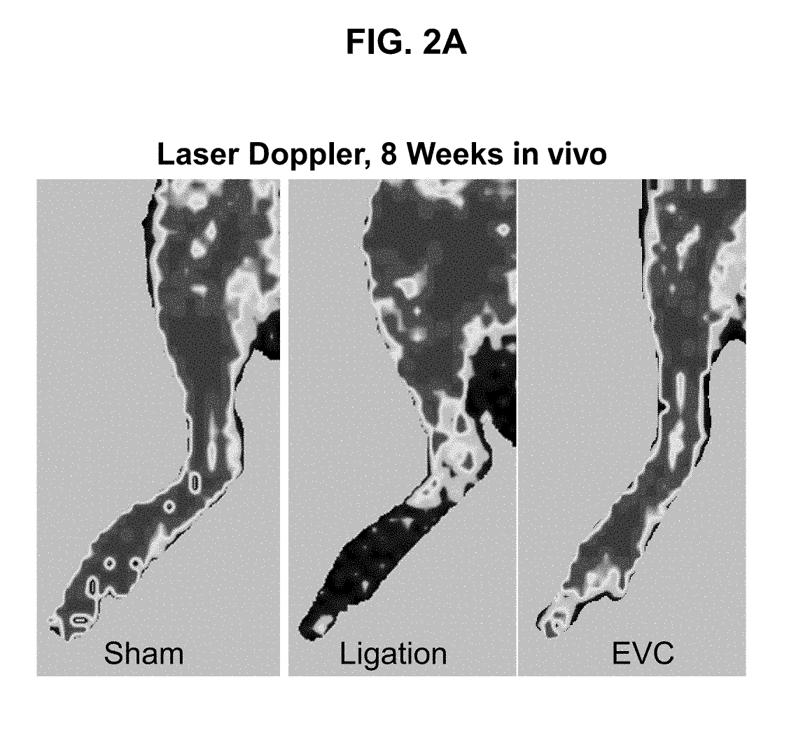

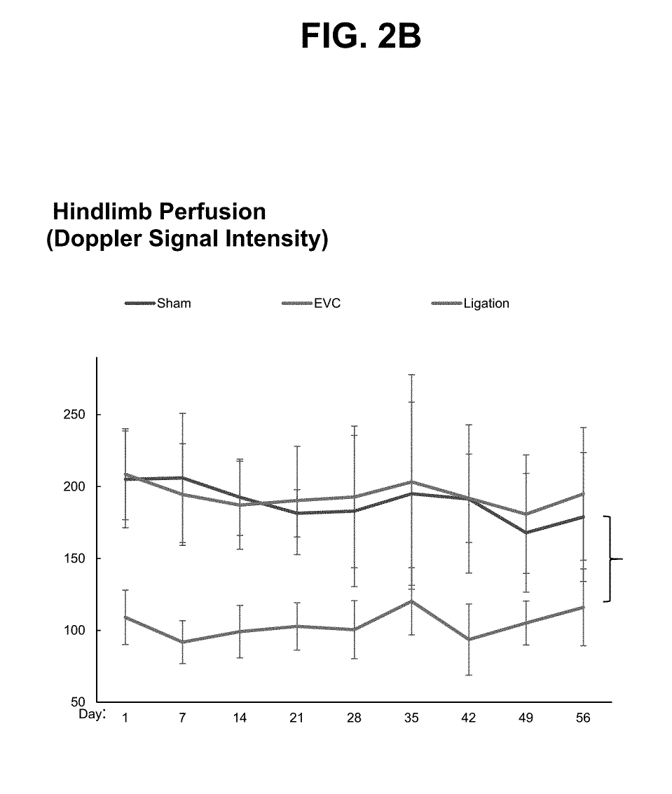

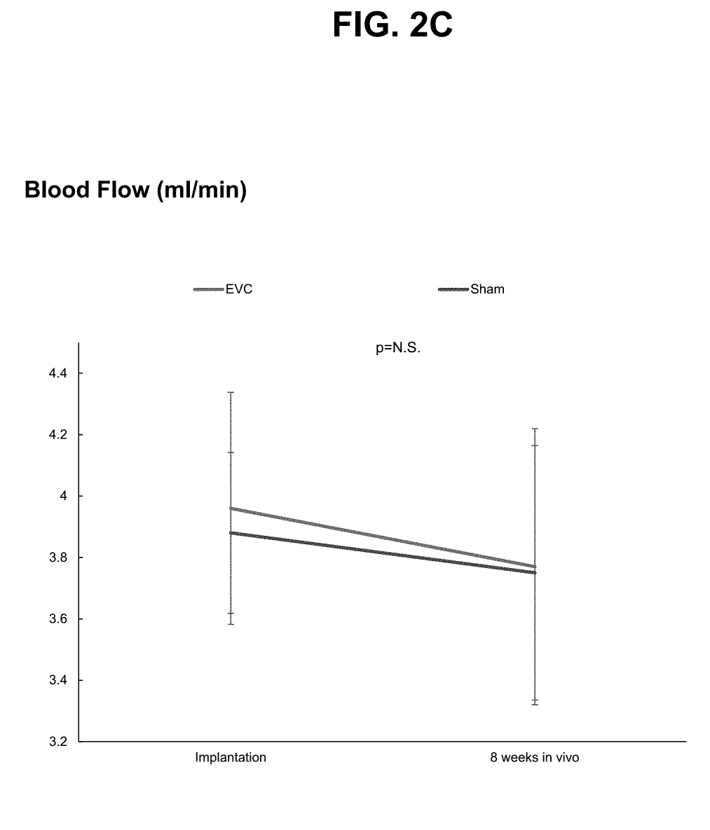

Here, we describe a technique of tissue engineering blood vessels that works with a temporary scaffold, thereby combining the advantages of high patency rates and rapid production. We have demonstrated the rapid construction of small vessels that are later anastomosed to the femoral artery as an interpositional vessel graft (FIGS. 1A and 1B). First, human fibroblasts and smooth muscle cells were seeded in two layers on 3.5 cm temperature responsive cell-culture dishes. They formed a confluent cell sheet that could be lifted after 24 hours upon temperature reduction. Between five and ten individual cell sheets were wrapped around a 22.5 gauge angiocath needle. To stabilize the cell sheets, cyanoacrylate membranes were added (cyanoacrylate can be replaced by fibrin glue, if necessary). These constructs were endothelialized with human umbilical vein endothelial cells. Next, the constructs were incubated in a peristaltic bioreactor and perfused with endothelial cell growth media at 0.1 ml/minute for 10-14 days. To evaluate the alignment of the endothelial cells and the vessel wall orientation, engineered arteries were cryopreserved and analyzed histologically with hematoxylin and eosin staining (FIG. 1C). Immunofluorescence was also used with anti-von Willebrand Factor (vWF) antibody to visualize endothelial cells and with anti-α-smooth muscle actin (α-SMA) antibody to visualize smooth muscle cell alignment (FIG. 1D). Vessel patency was evaluated in a hindleg ischemia model, which demonstrated significantly improved perfusion after femoral artery ligation when the engineered vessel was used as an interposition graft (FIG. 2).

### Advantages and Improvements

The described technology utilizes cyanoacrylate as a temporary scaffold and thereby combines the advantages of a fast production process and high patency rates after implantation. A major advantage over previous methods of producing cell-sheet based vessel grafts is the short construction time of 10-14 days as opposed to 10-15 weeks. The reason is that the membrane stabilizes the graft so that it can be perfused immediately which significantly accelerates the maturation of the vessel wall. Cyanoacrylate has been tested extensively for various applications, including sealing the skin, controlling internal bleedings and anastomosing small vessels in plastic surgery. Additionally, the mechanical properties of the engineered graft resemble those of native human vessels so that they can be used for surgical anastomosis.

The use of cell sheet technology to engineer the vascular system prevents any contact of artificial scaffolds with the blood stream. The artificial scaffold used, cyanoacrylate, is added in thin membranes around the cell sheets, so that the endothelial layer and the smooth muscle cell layer consist of cells and biological matrix only. This is further substantiated by our preliminary data where the engineered arteries are patent for 8 weeks without anti-coagulation.

### References

Eagle K A, Guyton R A, Davidoff R, Edwards F H et al. ACC/AHA 2004 guideline update for coronary artery bypass graft surgery: a report of the American College of Cardiology/American Heart Association Task Force on Practice Guidelines. Circulation. 2004 Oct. 5;110(14):e340-437.

Samand Pashneh-Tala, Sheila MacNeil, and Frederik Claeyssens. The Tissue-Engineered Vascular Graft—Past, Present, and Future. Tissue Eng Part B Rev. 2016 Feb. 1; 22(1): 68-100.

McAllister T N, Maruszewski M, Garrido S A, Wystrychowski W et al. Effectiveness of haemodialysis access with an autologous tissue-engineered vascular graft: a multicentre cohort study. Lancet. 2009 Apr. 25;373(9673):1440-6.

C. E. Fernandez, R. W. Yen, S. M. Perez, H. W. Bedell et al. Human Vascular Microphysiological System for in vitro Drug Screening. Sci Rep. 2016; 6: 21579.

Youngmee Jung, HaYeun Ji, Zaozao Chen, Hon Fai Chan et al. Scaffold-free, Human Mesenchymal Stem Cell-Based Tissue Engineered Blood Vessels. Scientific Reports 2015; 5; 15116.

Zeng X Q, Ma L L, Tseng Y J, Chen J et al. Endoscopic cyanoacrylate injection with or without lauromacrogol for gastric varices: A randomized pilot study. J Gastroenterol Hepatol. 2017 March;32(3):631-638.

Silvestri A, Brandi C, Grimaldi L, Nisi G et al. Octyl-2-cyanoacrylate adhesive for skin closure and prevention of infection in plastic surgery. Aesthetic Plast Surg. 2006 November-December; 30(6):695-9.

Chang E I, Galvez M G, Glotzbach J P, Hamou C D. Vascular anastomosis using controlled phase transitions in poloxamer gels. Nat Med. 2011 Aug. 28;17(9):1147-52.

### EXAMPLE 2

### Engineering Perfused Human Heart Muscle to Remuscularize Ischemic Myocardium

### Background:

Regenerative cell therapies have shown promise in restoring heart function and facilitating structural repair following myocardial injury in various experimental studies. These beneficial effects are mainly mediated through the paracrine effects of pro-angiogenic cytokines released from the transplanted cells. Recent studies have focused on the transplantation of spatially organized cell patches containing cardiomyocytes, fibroblasts, and endothelial cells with the goal of enhancing cytokine secretion and enabling structural integration of donated cells into the native heart muscle.

Despite multiple approaches to engineer spatially oriented and pre-vascularized heart muscle patches, the central goal of regenerative cell therapies—significant remuscularization via structural integration of donated cardiomyocytes—remains elusive. Moreover, the thickness, viability and survival of the cell grafts are critically limited. One likely explanation for these shortcomings is the lack of an adequate blood supply immediately following cell transplantation. As a result, the cell grafts become ischemic and depend on the diffusion of nutrients from an equally ischemic post-infarction region of the host myocardium. The lack of an adequate blood supply and competition for nutrients constitutes a vicious cycle that inhibits the integration of cardiomyocytes and thereby the remuscularization of the infarcted myocardium.

The proposed solution is a pre-vascularized heart muscle transplant that can be directly anastomosed to the host circulation. This project consists of three major steps, i) the construction of an engineered artery as a main vessel for proximal and distal anastomoses, ii) the cultivation of vascularized engineered heart muscle around the main vessel and iii) the implantation of the heart muscle transplant in a rat model of myocardial infarction.

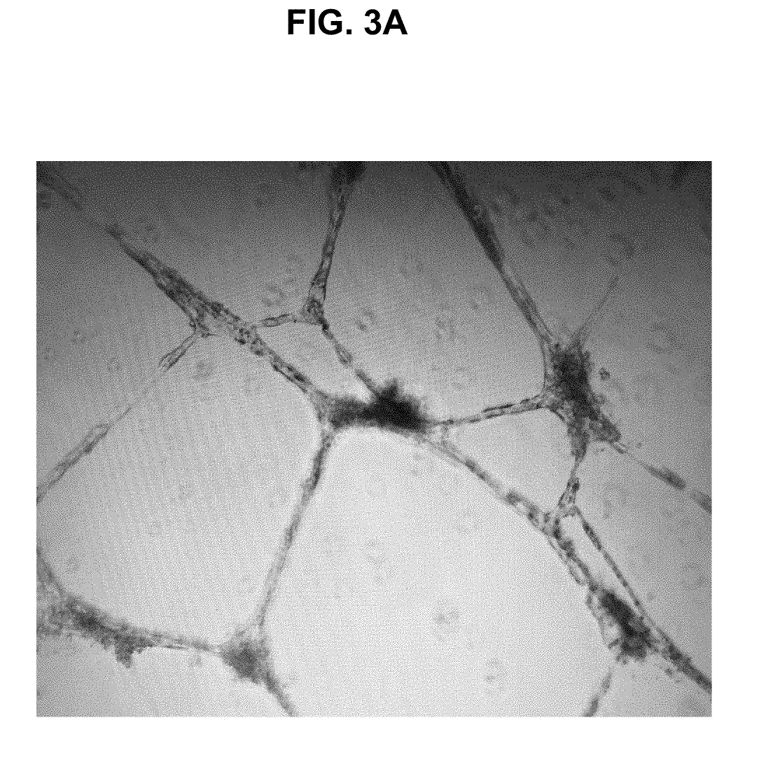

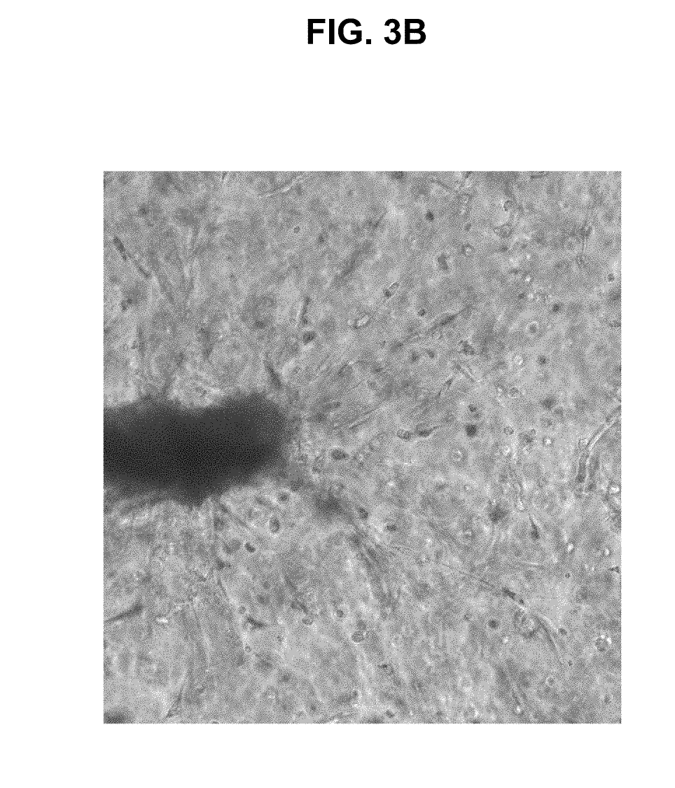

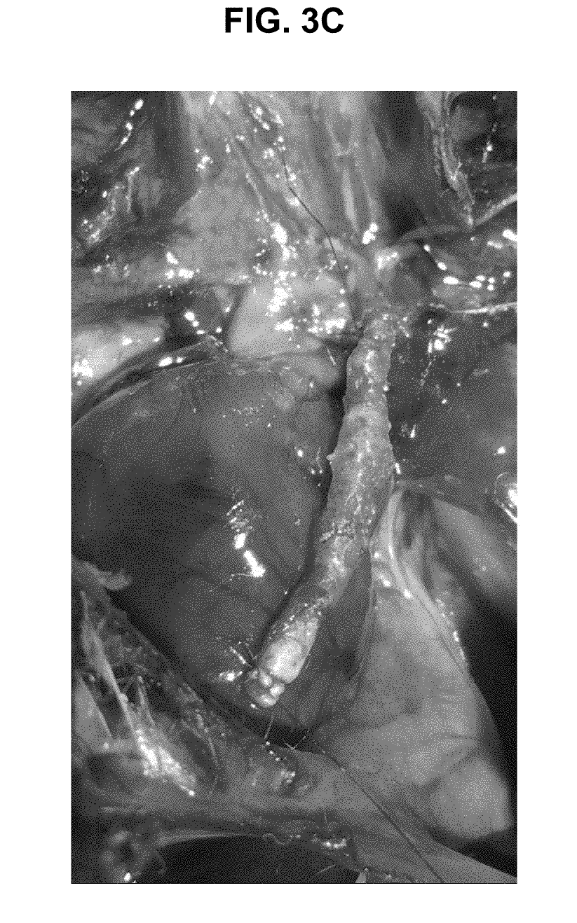

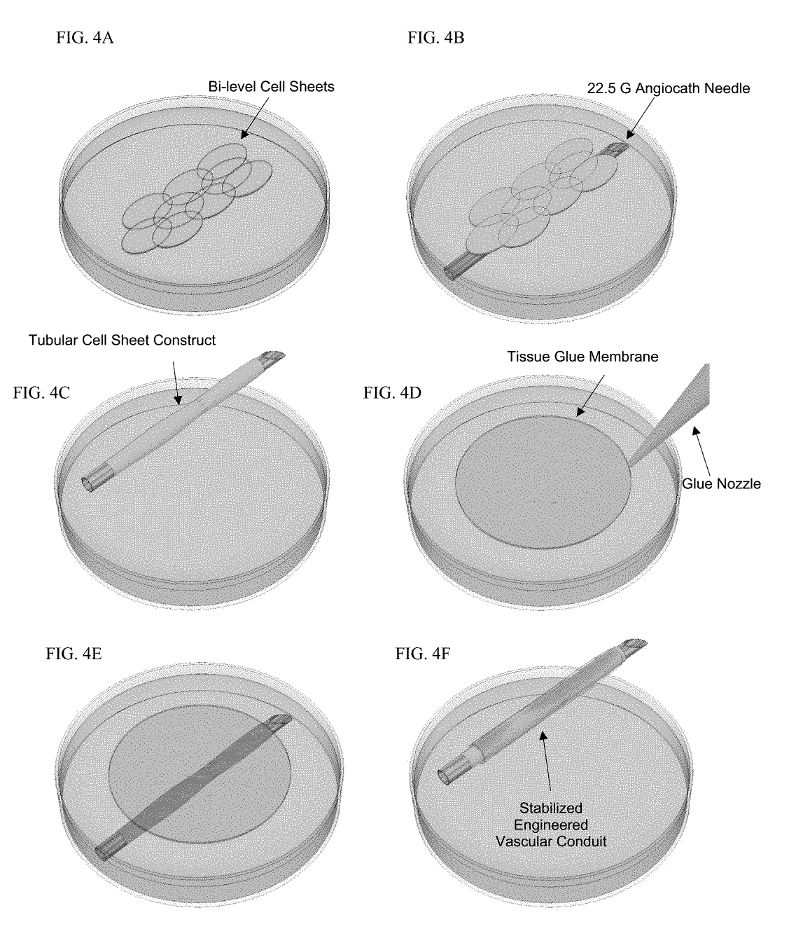

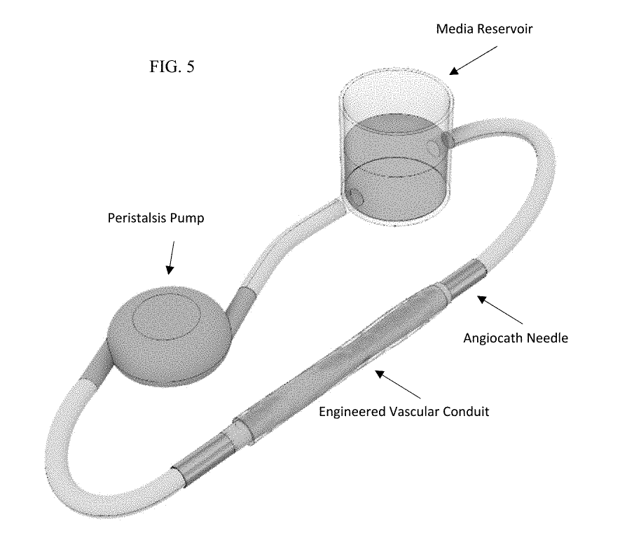

Engineering of a perfused heart muscle transplant. We have demonstrated the ability to rapidly construct small vessels (Example 1). These vessels can be used to anastomose engineered heart tissue to the host circulation (FIGS. 1A and 1B). The first step in constructing a perfused heart muscle transplant is the co-culture of the engineered vessel and human induced cardiomyocytes (FIG. 3B). The central part of the vessel, which is later transplanted to ischemic cardiac tissue following myocardial infarction, is placed on an 11 mm well on top of 0.2 ml of polymerized collagen hydrogel, which contains 4 million induced cardiomyocytes per ml (FIG. 3B). After one week, intensive vessel sprouting from the engineered vessel into the cardiomyocyte patch was visible (FIG. 3B).

This vascular network is further enhanced and organized by induction of flow through the engineered vessel at 0.1 ml/minute in a peristaltic bioreactor. Lectin is used to visualize the vascular network, and immunofluorescence is used for further analysis of the vascular network and graft, with von-Willebrand-Factor staining for endothelial cells, α-smooth muscle-actin staining for smooth muscle cells, cardiac troponin staining for cardiomyocytes, and α-actinin staining for Z-line formation. Mechanical testing is performed on the engineered heart muscle with atomic force microscopy and Instron, which is compared to healthy and post-myocardial infarction native tissue samples.

Implantation of the perfused heart muscle graft after myocardial infarction. A rodent model of myocardial infarction with left anterior descending coronary artery ligation on homozygous nude rats is used. The engineered heart muscle graft is anastomosed proximally to the left common carotid artery (FIG. 3C), sutured to the infarcted left ventricle, and distally anastomosed to the left internal mammary artery. Three control groups are included: (1) post-infarction animals receiving a non-perfused transplanted heart muscle patch, (2) animals undergoing LAD occlusion without any treatment and (3) animals with sham surgery alone. Vessel patency and left ventricular function is assessed after two weeks via echocardiography. The study endpoint is at six weeks, at which time heart function is assessed via left heart catheterization and cardiac MRI. Engineered vessel flow is assessed with an ultrasonic flow probe.

Next, animals undergo lectin perfusion via the jugular vein followed by euthanasia and heart explantation; specimens are cryopreserved. Lectin allows for visualization of the functional vessels within the graft and host tissue. Histology and immunofluorescence is performed as described above with additional stains including integrin β1 to assess for cell matrix attachment. Lastly, fate tracking is performed, using signal regulatory protein alpha (specific to human induced-cardiomyocytes), to determine survival and integration of transplanted cardiomyocytes.

### References:

Shudo Y, Cohen J E, Macarthur J W, Atluri P, et al. Spatially oriented, temporally sequential smooth muscle cell-endothelial progenitor cell bi-level cell sheet neovascularizes ischemic myocardium. Circulation. 2013 Sep. 10;128(11 Suppl 1):559-68.

Weinberger F, Breckwoldt K, Pecha S, Kelly A, et al. Cardiac repair in guinea pigs with human engineered heart tissue from induced pluripotent stem cells. Sci Transl Med. 2016 Nov. 2;8(363):363ra148.

Riegler J, Tiburcy M, Ebert A, Tzatzalos E, et al. Human Engineered Heart Muscles Engraft and Survive Long Term in a Rodent Myocardial Infarction Model. Circ Res. 2015 Sep. 25; 117(8):720-30.

Tiburcy M, Hudson J E, Balfanz P, Schlick S, et al. Defined Engineered Human Myocardium With Advanced Maturation for Applications in Heart Failure Modeling and Repair. Circulation. 2017 May 9; 135(19):1832-1847.

Riemenschneider S B, Mattia D J, Wendel J S, Schaefer J A, et al. Inosculation and perfusion of prevascularized tissue patches containing aligned human microvessels after myocardial infarction. Biomaterials. 2016 August;97:51-61.

Kawamura M, Miyagawa S, Fukushima S, Saito A, et al. Enhanced survival of transplanted human induced pluripotent stem cell-derived cardiomyocytes by the combination of cell sheets with the pedicled omental flap technique in a porcine heart. Circulation. 2013 Sep. 10;128(11 Suppl 1):S87-94.

Karakikes I, Ameen M, Termglinchan V, Wu J C. Human induced pluripotent stem cell-derived cardiomyocytes: insights into molecular, cellular, and functional phenotypes. Circ Res. 2015 Jun. 19;117(1):80-8.

L'Heureux N, Dusserre N, Konig G, Victor B, et al. Human tissue-engineered blood vessels for adult arterial revascularization. Nat Med. 2006 March; 12(3):361-5.

While the preferred embodiments of the invention have been illustrated and described, it will be appreciated that various changes can be made therein without departing from the spirit and scope of the invention.

What is claimed is:

1. A method of making a tissue-engineered blood vessel comprising:
a) culturing fibroblasts and smooth muscle cells to form one or more confluent cell sheets;
b) wrapping said one or more cell sheets around a rod-like device to form a tube;
c) stabilizing the tube formed from the cell sheets with a cyanoacrylate membrane or fibrin glue;
d) endothelialization of the tube formed from the cell sheets by culturing with endothelial cells; and
e) removing the rod-like device to form the tissue-engineered blood vessel.

2. The method of claim 1, wherein the fibroblasts and smooth muscle cells are from a human subject.

3. The method of claim 1, wherein the endothelial cells are human umbilical vein endothelial cells.

4. The method of claim 1, wherein the diameter of the rod-like device is less than or equal to 1 mm.

5. The method of claim 1, wherein the rod-like device is a mandrel or needle.

6. The method of claim 5, wherein the needle is an angiocath needle.

7. The method of claim 5, wherein the needle has a gauge of at least 11, at least 16, at least 18, at least 20, at least 22, or at least 22.5.

8. The method of claim 7, wherein the needle has a gauge ranging from 11 to 24.

9. The method of claim 8, wherein the needle has a gauge of 22.5.

10. The method of claim 1, wherein at least 4, 5, 6, 7, 8, 9, 10, 11, or 12 cell sheets are wrapped around the rod-like device to form the tube.

11. The method of claim 1, wherein 5 to 10 cell sheets are wrapped around the rod-like device to form the tube.

12. A tissue-engineered blood vessel produced by the method of claim 1.

13. The tissue-engineered blood vessel of claim 12, wherein the diameter of the tissue-engineered blood vessel is less than or equal to 1 mm.

14. A method of treating a subject fora cardiovascular disease or disorder, the method comprising implanting the tissue-engineered blood vessel of claim 12 in the subject.

15. The method of claim 14, further comprising linking the tissue-engineered blood vessel to a vein or artery by surgical anastomosis.

16. The method of claim 14, wherein the tissue-engineered blood vessel is used in a vascular bypass or interposition graft.

17. The method of claim 14, wherein the cardiovascular disease or disorder is selected from the group consisting of coronary artery disease, ischemic heart disease, ischemic stroke, peripheral artery disease, cerebrovascular disease, atherosclerosis, arteriosclerosis, angina, myocardial infarction, and embolism.

18. The method of claim 14, wherein the fibroblasts, smooth muscle cells, or endothelial cells are autologous, xenogeneic, or allogeneic.

19. A method of vascularizing a tissue or organ for transplant, the method comprising implanting the tissue-engineered blood vessel of claim 12 in the tissue or organ.

20. The method of claim 19, wherein the tissue is heart muscle.

21. The method of claim 19, wherein said implanting is performed prior to or after transplant of the tissue or organ into a subject.

22. The method of claim 19, further comprising cultivation of the tissue around the tissue-engineered blood vessel.

23. The method of claim 19, further comprising perfusing the vascularized tissue wherein blood flows through the tissue-engineered blood vessel.

24. The method of claim 19, further comprising transplanting the vascularized tissue or organ into a subject.

25. The method of claim 19, further comprising linking the tissue-engineered blood vessel to a vein or artery by surgical anastomosis.

26. A method of engineering a perfused heart muscle tissue graft, the method comprising:
a) producing a tissue-engineered blood vessel according to the method of claim 1;
b) co-culturing the tissue-engineered blood vessel with cardiomyocytes to produce a vascularized heart muscle tissue graft, wherein vessel sprouting from the tissue-engineered blood vessel produces a vascular network within the heart muscle tissue graft; and
c) perfusing the cardiomyocyte tissue graft, wherein blood flows through the tissue-engineered blood vessel and the vascular network within the heart muscle tissue graft.

27. The method of claim 26, wherein the cardiomyocytes are autologous, xenogeneic, or allogeneic.

28. The method of claim 26, wherein the cardiomyocytes are human induced cardiomyocytes.
